(19) United States
(12) Patent Application Publication
(10) Pub. No.: US 2025/0145551 A1
(43) Pub. Date: May 8, 2025

(54) SYNTHESIS OF HFO-153-10MCZZ INCLUDING CATALYTIC COUPLING OF HCFC-225CA OR CFC-215CB

(71) Applicant: THE CHEMOURS COMPANY FC, LLC, WILMINGTON, DE (US)

(72) Inventors: XUEHUI SUN, KENNETT SQUARE, PA (US); DREW RICHARD BRANDT, WEST CHESTER, PA (US); ROBERT DANIEL LOUSENBERG, WILMINGTON, DE (US); BARBARA HAVILAND MINOR, THE VILLAGES, FL (US)

(73) Assignee: THE CHEMOURS COMPANY FC, LLC, WILMINGTON, DE (US)

(21) Appl. No.: 18/836,840

(22) PCT Filed: Feb. 24, 2023
(86) PCT No.: PCT/US2023/013768

§ 371 Date: Aug. 8, 2024

Related U.S. Application Data
(60) Provisional application No. 63/313,773, filed on Feb. 25, 2022.

(51) Int. Cl. C07C 17/20

(52) U.S. Cl. CPC C07C 17/20

(57) ABSTRACT

In an embodiment, a method of producing a fluoroolefin comprises coupling 3,3-dichloro-1,1,1,2,2-pentafluoropropane (CF₃—CF₂—CHCl₂) in a liquid phase in the presence of a catalyst to form a composition comprising 1,1,1,2,2,5,5,6,6,6-decafluoro-3-hexene (CF₃CF₂CH═CHCF₂CF₃). In another embodiment, a method of producing a fluoroolefin comprises coupling 1,1,1-trichloropentafluoropropane (CF₃—CF₂—CCl₃) in a vapor phase in the presence of a first catalyst to form a composition comprising 3,4-dichloro-1,1,1,2,2,5,5,6,6,6-decafluoro-3-hexene (CF₃—CF₂—CCl═CCl—CF₂—CF₃). The method may further include forming the CF₃—CF₂—CCl₃ in a vapor phase from CF₃CF₂CHₘCl₃₋ₘ and chlorine (Cl₂). The method may further include hydrodechlorinating the 3,4-dichloro-1,1,1,2,2,5,5,6,6,6-decafluoro-3-hexene with hydrogen (H₂) in a vapor phase in the presence of a second catalyst to form a composition comprising CF₃CF₂CH═CHCF₂CF₃ and 3-chloro-1,1,1,2,2,5,5,6,6,6-decafluoro-3-hexene (CF₃CF₂CCl═CHCF₂CF₃).

This application claims the benefit of Application No. 63/313,773, filed on Feb. 25, 2022. The disclosure of Application No. 63/313,773 is hereby incorporated by reference in its entirety.

### FIELD

The present disclosure is directed to the production of fluorinated alkene compounds. More specifically, the present disclosure is directed to the production of the hydrofluoroolefin (HFO) 1,1,1,2,2,5,5,6,6,6-decafluoro-3-hexene (HFO-153-10mczz; CF₃CF₂CH═CHCF₂CF₃).

### BACKGROUND

A growing public awareness of the environmental impacts from the extraction, transportation, and use of fossil fuels is motivating a new environmental sustainability driver in the form of regulations and reduction in output of CO₂ equivalents in the atmosphere. New working fluids with a low global warming potential (GWP) and a low ozone depletion potential (ODP) for both existing and new applications in thermal management segments will need to adhere to these new regulations.

### SUMMARY

In an embodiment, a method of producing a fluoroolefin comprises coupling 3,3-dichloro-1,1,1,2,2-pentafluoropropane (CF₃—CF₂—CHCl₂) in a liquid phase in the presence of a catalyst to form a composition comprising 1,1,1,2,2,5,5,6,6,6-decafluoro-3-hexene (HFO-153-10mczz; CF₃CF₂CH═CHCF₂CF₃).

In another embodiment, a method of producing a fluoroolefin comprises coupling 1,1,1-trichloropentafluoropropane (CF₃—CF₂—CCl₃) in a vapor phase in the presence of a first catalyst to form a composition comprising 3,4-dichloro-1,1,1,2,2,5,5,6,6,6-decafluoro-3-hexene (CF₃—CF₂—CCl═CCl—CF₂—CF₃).

In yet another embodiment, a method of producing a fluoroolefin comprises forming CF₃—CF₂—CCl₃ in a vapor phase from CF₃CF₂CHₙCl₃₋ₙ and chlorine (Cl₂); wherein n is an integer selected from the group consisting of 1, 2, and 3.

In yet another embodiment, a method of producing a fluoroolefin comprises coupling a compound of formula (1), C₂F₅CHₙCl₃₋ₙ (1) wherein n is 0 or 1; in the presence of a first catalyst to form a composition comprising a compound of formula (2), C₂F₅CX₁=CX₂C₂F₅ (2) wherein when n is 0, X₁ and X₂ are Cl; and wherein when n is 1, X₁ and X₂ are H.

The present disclosure includes the following aspects and embodiments:
In one embodiment, disclosed herein are methods of producing a fluoroolefin. The methods described herein include coupling 3,3-dichloro-1,1,1,2,2-pentafluoropropane (CF₃—CF₂—CHCl₂) in a liquid phase in the presence of a catalyst to form a composition comprising 1,1,1,2,2,5,5,6,6,6-decafluoro-3-hexene (CF₃CF₂CH═CHCF₂CF₃).

According to the foregoing embodiment, also disclosed herein are methods wherein the coupling occurs in an aprotic solvent.

According to the foregoing embodiment, also disclosed herein are methods wherein the aprotic solvent is selected from the group consisting of dimethylformamide, dimethylacetamide, dimethyl sulfoxide, and N-methylpyrrolidone.

According to any of the foregoing embodiments, also disclosed herein are methods wherein the catalyst is selected from the group consisting of 2,2-bipyridine, a copper(I) salt, and a combination thereof.

According to the foregoing embodiment, also disclosed herein are methods wherein the copper(I) salt is selected from the group consisting of CuCl, CuBr, CuI, and copper(I) acetate.

According to any of the foregoing embodiments, also disclosed herein are methods wherein the coupling occurs in the presence of copper powder.

In one embodiment, disclosed herein are methods of producing a fluoroolefin. The methods described herein include coupling 1,1,1-trichloropentafluoropropane (CF₃—CF₂—CCl₃) in a vapor phase in the presence of a first catalyst to form a composition comprising 3,4-dichloro-1,1,1,2,2,5,5,6,6,6-decafluoro-3-hexene (CF₃—CF₂—CCl═CCl—CF₂—CF₃).

According to the foregoing embodiment, also disclosed herein are methods wherein the first catalyst comprises Ru/SiC.

According to any of the foregoing embodiments, also disclosed herein are methods further comprising forming the CF₃—CF₂—CCl₃ in a vapor phase from CF₃CF₂CHₙCl₃₋ₙ and chlorine (Cl₂), wherein n is an integer selected from the group consisting of 1, 2, and 3. In some embodiments, the forming occurs in the absence of a catalyst. In other embodiments, the forming occurs in the presence of a catalyst selected from the group consisting of an activated carbon catalyst, a metal halide catalyst, a metal oxide catalyst, a metal oxyhalide catalyst, and combinations thereof.

According to any of the foregoing embodiments, also disclosed herein are methods further comprising hydrodechlorinating the 3,4-dichloro-1,1,1,2,2,5,5,6,6,6-decafluoro-3-hexene with hydrogen (H₂) in a vapor phase in the presence of a second catalyst to form a composition comprising 1,1,1,2,2,5,5,6,6,6-decafluoro-3-hexene (CF₃CF₂CH═CHCF₂CF₃) and 3-chloro-1,1,1,2,2,5,5,6,6,6-decafluoro-3-hexene (CF₃CF₂CCl═CHCF₂CF₃).

According to the foregoing embodiment, also disclosed herein are methods wherein the second catalyst comprises a nickel-containing catalyst.

According to the foregoing embodiment, also disclosed herein are methods wherein the second catalyst comprises an iridium/carbon catalyst.

According to the foregoing embodiment, also disclosed herein are methods wherein the second catalyst comprises a bimetallic catalyst on a carbon support.

According to any of the foregoing embodiments, also disclosed herein are methods further comprising hydrodechlorinating the 3-chloro-1,1,1,2,2,5,5,6,6,6-decafluoro-3-hexene (CF₃CF₂CCl═CHCF₂CF₃) with hydrogen (H₂) in a vapor phase in the presence of a third catalyst to form 1,1,1,2,2,5,5,6,6,6-decafluoro-3-hexene (CF₃CF₂CH═CHCF₂CF₃).

According to the forgoing embodiment, also disclosed herein are methods wherein the third catalyst is selected from the group consisting of a nickel-containing catalyst, an iridium/carbon catalyst, a bimetallic catalyst on a carbon support, a gold catalyst on a support, a platinum catalyst on a support, a palladium catalyst on a support, a copper catalyst on a support, and combinations thereof.

According to any of the foregoing embodiments, also disclosed herein are methods wherein the coupling occurs in the presence of copper powder.

In one embodiment, disclosed herein are methods of producing a fluoroolefin. The methods described herein include forming CF₃—CF₂—CCl₃ in a vapor phase from CF₃CF₂CHₙCl₃₋ₙ and chlorine (Cl₂); wherein n is an integer selected from the group consisting of 1, 2, and 3.

According to the foregoing embodiment, also disclosed herein are methods wherein the forming occurs in the absence of a catalyst.

According to the foregoing embodiment, also disclosed herein are methods wherein n in 1.

According to the foregoing embodiment, also disclosed herein are methods wherein n in 3.

In one embodiment, disclosed herein are methods of producing a fluoroolefin. The methods described herein include coupling a compound of formula (1), C₂F₅CHₙCl₃₋ₙ (1) wherein n is 0 or 1; in the presence of a first catalyst to form a composition comprising a compound of formula (2), C₂F₅CX₁═CX₂C₂F₅ (2) wherein when n is 0, X₁ and X₂ are Cl; and wherein when n is 1, X₁ and X₂ are H.

According to the foregoing embodiment, also disclosed herein are methods wherein n is 1 and X₁ and X₂ are H, and the coupling occurs in a liquid phase.

According to the foregoing embodiment, also disclosed herein are methods wherein the coupling occurs in an aprotic solvent.

According to the foregoing embodiment, also disclosed herein are methods wherein the aprotic solvent is selected from the group consisting of dimethylformamide, dimethylacetamide, dimethyl sulfoxide, and N-methylpyrrolidone.

According to any of the foregoing embodiments, also disclosed herein are methods wherein the first catalyst is selected from the group consisting of 2,2-bipyridine, a copper(I) salt, and a combination thereof.

According to the foregoing embodiment, also disclosed herein are methods wherein the copper(I) salt is selected from the group consisting of CuCl, CuBr, CuI, and copper(I) acetate.

According to any of the foregoing embodiments, also disclosed herein are methods wherein the coupling occurs in the presence of copper powder.

According to any of the foregoing embodiments, also disclosed herein are methods wherein n is 0 and X₁ and X₂ are Cl, and the coupling occurs in a vapor phase.

According to the foregoing embodiment, also disclosed herein are methods wherein the first catalyst comprises Ru/SiC.

According to the foregoing embodiment, also disclosed herein are methods further comprising forming the CF₃—CF₂—CCl₃ in a vapor phase from CF₃CF₂CHₘCl₃₋ₘ and chlorine (Cl₂) wherein m is an integer selected from the group consisting of 1, 2, and 3. In some embodiments, the forming occurs in the absence of a catalyst. In other embodiments, the forming occurs in the presence of a catalyst selected from the group consisting of an activated carbon catalyst, a metal halide catalyst, a metal oxide catalyst, a metal oxyhalide catalyst, and combinations thereof.

According to the foregoing embodiment, also disclosed herein are methods further comprising hydrodechlorinating the 3,4-dichloro-1,1,1,2,2,5,5,6,6,6-decafluoro-3-hexene with hydrogen (H₂) in a vapor phase in the presence of a second catalyst to form a composition comprising 1,1,1,2,2,5,5,6,6,6-decafluoro-3-hexene (CF₃CF₂CH═CHCF₂CF₃) and 3-chloro-1,1,1,2,2,5,5,6,6,6-decafluoro-3-hexene (CF₃CF₂CCl═CHCF₂CF₃).

According to the foregoing embodiment, also disclosed herein are methods wherein the second catalyst is selected from the group consisting of a nickel-containing catalyst, an iridium/carbon catalyst, and a bimetallic catalyst on a carbon support.

According to any of the foregoing embodiments, also disclosed herein are methods further comprising hydrodechlorinating the 3-chloro-1,1,1,2,2,5,5,6,6,6-decafluoro-3-hexene (CF₃CF₂CCl═CHCF₂CF₃) with hydrogen (H₂) in a vapor phase in the presence of a third catalyst to form 1,1,1,2,2,5,5,6,6,6-decafluoro-3-hexene (CF₃CF₂CH═CHCF₂CF₃).

According to the forgoing embodiment, also disclosed herein are methods wherein the third catalyst is selected from the group consisting of a nickel-containing catalyst, an iridium/carbon catalyst, a bimetallic catalyst on a carbon support, a gold catalyst on a support, a platinum catalyst on a support, a palladium catalyst on a support, a copper catalyst on a support, and combinations thereof.

According to any of the foregoing embodiments, also disclosed herein are compositions formed by any of the foregoing methods.

According to certain embodiments of the invention fluoroolefins E-C₂F₅CF═CFC₂F₅(E-HFO-151-12mcyy), E-C₂F₅CH═CHC₂F₅, (E-HFO-153-10mczz), C₃F₇CH═CHCF₃ (HFO-153-10mzz), (CF₃)₂CFCH═CHCF₃ (HFO-153-10mzzy), Z—C₂F₅CF═CFC₂F₅ (z-FO-151-12mcyZ) are HFO-153-10mzcc is used for thermal energy management of electrical components.

According to other embodiments, fluoroolefins comprising E-C₂F₅CF═CFC₂F₅ (E-HFO-151-12mcyy), E-C₂F₅CH═CHC₂F₅, (E-HFO-153-10mczz), C₃F₇CH═CHCF₃ (HFO-153-10mzz), (CF₃)₂CFCH═CHCF₃ (HFO-153-10mzzy), Z—C₂F₅CF═CFC₂F₅ (z-FO-151-12mcyZ) are used with the electrical components requiring thermal management such as electronics, computers, servers and data centers, and electronic components, including, but not limited to, batteries, electric motors, heat transfer components found in one of hybrid electric vehicles (HEV), mild hybrids electric vehicles (M HEV), plug-in hybrid electric vehicles (PHEV), or electric vehicles (EV), heat pump systems of these vehicles, and electrified mass transport.

Other features and advantages of the present invention will be apparent from the following more detailed description, which illustrates, by way of example, the principles of the invention.

### BRIEF DESCRIPTION OF THE DRAWINGS

FIG. 1 is a schematic in which one of the fluoroolefins comprising E/Z fluoroolefins described herein is used as a thermal fluid being circulated.

FIG. 2 is a schematic of one immersion cooling embodiment, using the E/Z fluoroolefins described herein.

### DETAILED DESCRIPTION

Provided are synthesis methods to produce fluorinated alkenes which comprise, consist essentially of or consist of one of E-C₂F₅CF═CFC₂F₅(E-HFO-151-12mcyy), E-C₂F₅CH═CHC₂F₅, (E-HFO-153-10mczz), C₃F₇CH═CHCF₃ (HFO-153-10mzz), (CF₃)₂CFCH═CHCF₃ (HFO-153-10mzzy) or Z—C₂F₅CF═CFC₂F₅ (z-FO-151-12mcyZ), which can be used alone or in blends.

As used herein, the terms “comprises,” “comprising,” “includes,” “including,” “has,” “having” or any other variation thereof, are intended to cover a non-exclusive inclusion. For example, a composition, process, method, article, or apparatus that comprises a list of elements is not necessarily limited to only those elements but may include other elements not expressly listed or inherent to such composition, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

The transitional phrase “consisting of” excludes any element, step, or ingredient not specified. If in the claim such would close the claim to the inclusion of materials other than those recited except for impurities ordinarily associated therewith. When the phrase “consists of” appears in a clause of the body of a claim, rather than immediately following the preamble, it limits only the element set forth in that clause; other elements are not excluded from the claim as a whole.

The transitional phrase “consisting essentially of” is used to define a composition, method that includes materials, steps, features, components, or elements, in addition to those literally disclosed provided that these additional included materials, steps, features, components, or elements do not materially affect the basic and novel characteristic(s) of the claimed invention, especially the mode of action to achieve the desired result of any of the processes of the present invention. The term “consisting essentially of” occupies a middle ground between “comprising” and “consisting of”.

Provided are synthesis methods to produce fluorinated alkenes, which overcome the limitations described above. Disclosed herein are synthetic routes to form 1,1,1,2,2,5,5,6,6,6-decafluoro-3-hexene (HFO-153-10mczz; CF₃CF₂CH═CHCF₂CF₃).

Embodiments of the present disclosure, for example, provide methods to produce fluorinated alkenes. More specifically, the present disclosure provides methods to produce fluorinated alkenes having a perfluorinated alkyl chain. The resulting fluorinated alkenes are environmentally friendly, exhibiting a low GWP and zero ozone depletion potential (ODP), non-flammable, non-conductive, and exhibit low liquid viscosities.

Methods and compositions of the present disclosure may include one or more of 3,4-dichloro-1,1,1,2,2,5,5,6,6,6-decafluoro-3-hexene (CFO-151-10mcxx; CF₃—CF₂—CCl═CCl—CF₂—CF₃), 3-chloro-1,1,1,2,2,5,5,6,6,6-decafluoro-3-hexene (HCFO-152-10mcxz; CF₃CF₂CCl═CHCF₂CF₃), and/or 1,1,1,2,2,5,5,6,6,6-decafluoro-3-hexene (HFO-153-10mczz; CF₃CF₂CH═CHCF₂CF₃). Each of these compounds has an E isomeric form (E-CFO-151-10mcxx, E-HCFO-152-10mcxz, and E-HFO-153-10mczz, respectively) and a Z isomeric form (Z—CFO-151-10mcxx, Z—HCFO-152-10mcxz, and Z—HFO-153-10mczz, respectively). As used herein, when the form is unspecified, the composition may include the E isomer, the Z isomer, or any combination thereof.

In some embodiments, the E isomer is preferred. In some embodiments, starting materials and/or method conditions are selected to increase formation of the E isomer over the Z isomer. In some embodiments, the method includes separating the E isomer from the Z isomer.

A first method of producing a fluoroolefin includes coupling 3,3-dichloro-1,1,1,2,2-pentafluoropropane (HCFC-225ca; CF₃—CF₂—CHCl₂) in a liquid phase in a reactor in the presence of a catalyst to form a composition including 1,1,1,2,2,5,5,6,6,6-decafluoro-3-hexene (HFO-153-10mczz; CF₃CF₂CH═CHCF₂CF₃). In some embodiments, the reactor is a Hastelloy® shaker tube. In some embodiments, copper powder is also provided in the reactor.

In some embodiments, the coupling occurs at a temperature within the range of about 50° C. to about 140° C., alternatively about 60° C. to about 120° C., alternatively about 80° C. to about 120° C., or any value, range, or sub-range therebetween.

In some embodiments, the liquid phase coupling occurs in an aprotic solvent. Appropriate aprotic solvents may include, but are not limited to, dimethylformamide, dimethylacetamide, dimethyl sulfoxide, or N-methylpyrrolidone.

In some embodiments, the catalyst is 2,2-bipyridine. In other embodiments, the catalyst is a copper(I) salt. In other embodiment, the catalyst is a combination of 2,2-bipyridine and a copper(I) salt. Appropriate copper(I) salts may include, but are not limited to, CuCl, CuBr, CuI, and copper(I) acetate.

In some embodiments, the HCFC-225ca starting material also includes other fluorinated compounds, such as, for example, 1,3-dichloro-1,1,2,2,3-pentafluoropropane (HCFC-225cb; CClF₂—CF₂—CHClF) and/or 2,2-dichloro-1,1,1,3,3-pentafluoropropane (HCFC-225aa; CF₃—CCl₂—CHF₂).

In some embodiments, the first method further includes purifying the HFO-153-10mczz.

A second method of producing a fluoroolefin includes coupling 1,1,1-trichloropentafluoropropane (CFC-215cb; CF₃—CF₂—CCl₃) in a vapor phase in a reactor in the presence of a first catalyst to form a composition including 3,4-dichloro-1,1,1,2,2,5,5,6,6,6-decafluoro-3-hexene (CFO-151-10mcxx; CF₃—CF₂—CCl═CCl—CF₂—CF₃). In some embodiments, the reactor is an alloy tube reactor, such as, for example, a Monel® tube reactor.

In some embodiments, the first catalyst includes Ru/SiC. In some embodiments, hydrogen gas (H₂) is also supplied to the reactor.

In some embodiments, the vapor phase coupling occurs at a temperature in the range of about 100° C. to about 140° C., alternatively about 125° C. to about 135° C., alternatively about 127° C. to about 133° C., alternatively about 130° C., or any value, range, or subrange therebetween.

In some embodiments, the vapor phase coupling occurs at a pressure in the range of about 0 psig to about 175 psig, alternatively about 125 psig to about 170 psig, alternatively about 140 psig to about 160 psig, alternatively about 145 psig to about 155 psig, alternatively about 150 psig, or any value, range, or subrange therebetween.

In some embodiments, the vapor phase coupling occurs over a contact time period in the range of about 10 seconds hours to about 30 minutes. The contact time can also range from about 50 seconds to about 1 hour, about 70 seconds to 30 minutes and, in some cases, about 80 seconds to about 20 minutes.

In some embodiments, the second method also includes chlorinating a molecule of formula (2), CF₃CF₂CHₙCl₃₋ₙ, in the vapor phase in a reactor to form the CFC-215cb, where n is 1, 2, or 3. In some embodiments, the reactor is a metal alloy reactor. In some embodiments, the metal alloy reactor is an Inconel© tube. In some embodiments, the chlorinating occurs in the absence of a catalyst. In other embodiments, the chlorinating occurs in the presence of a catalyst. In some embodiments, the catalyst is an activated carbon catalyst, a metal halide catalyst, a metal oxide catalyst, and/or a metal oxyhalide catalyst, each of which may be provided either with or without a support. In some embodiments, the chlorinating occurs with ultraviolet (UV) radiation.

In some embodiments, the molecule of formula (2) is HCFC-225ca. In some embodiments, the HCFC-225ca starting material also includes other fluorinated compounds, such as, for example, HCFC-225cb, which becomes chlorinated to 1,1,3-trichloropentafluoropropane (CFC-215ca; CClF₂—CF₂—CCl₂F).

In some embodiments, the molecule of formula (2) is 1,1,1,2,2-pentafluoropropane 1,1,1,2,2-pentafluoro-3-chloropropane (HCFC-235cb; CF₃—CF₂—CH₂Cl).

In some embodiments, the molecule of formula (2) is 1,1,1,2,2-pentafluoropropane (HFC-245cb; CF₃—CF₂—CH₃).

In some embodiments, chlorine gas (Cl₂) is also supplied to the reactor for the chlorination reaction.

In some embodiments, the vapor phase chlorination occurs at a temperature in the range of about 200° C. to about 300° C.

In some embodiments, the vapor phase chlorination occurs over a contact time period in the range of about ten seconds to about two hours, alternatively about one minute to about one hour, alternatively about five minutes to about 30 minutes, or any value, range, or sub-range therebetween.

In some embodiments, the vapor phase chlorination occurs at a pressure in the range of about atmospheric (0 psig) to about 200 psig (about 1.38 mPa).

In some embodiments, the second method includes hydrodechlorinating the CFO-151-10mcxx with hydrogen (H₂) in a reactor in the vapor phase in the presence of a second catalyst to form a composition including HFO-153-10mczz and 3-chloro-1,1,1,2,2,5,5,6,6,6-decafluoro-3-hexene (CF₃CF₂CCl═CHCF₂CF₃).

In some embodiments, the second catalyst includes a nickel-containing catalyst. When the second catalyst includes a nickel-containing catalyst, the vapor phase hydrodechlorination preferably occurs over a contact time period in the range of about 50 seconds to about 80 seconds.

In some embodiments, the second catalyst includes an iridium/carbon catalyst. When the second catalyst includes an iridium/carbon catalyst, the vapor phase hydrodechlorination preferably occurs over a contact time period in the range of about 50 seconds to about 300 seconds.

In some embodiments, the second catalyst includes a bimetallic catalyst on a carbon support. When the second catalyst includes a bimetallic catalyst on a carbon support, the vapor phase hydrodechlorination preferably occurs over a contact time period in the range of about 50 seconds to about 130 seconds.

In some embodiments, the vapor phase hydrodechlorination occurs at a temperature in the range of about 70° C. to about 270° C., alternatively about 120° C. to about 270° C., alternatively about 120° C. to about 140° C., alternatively about 190° C. to about 210° C., alternatively about 240° C. to about 260° C., alternatively about 130° C., alternatively about 200° C., alternatively about 250° C., or any value, range, or subrange therebetween.

In some embodiments, the vapor phase hydrodechlorination occurs at a pressure in the range of about 0 psig to about 175 psig, alternatively about 130 psig to about 170 psig, alternatively about 140 psig to about 160 psig, alternatively about 145 psig to about 155 psig, alternatively about 150 psig, or any value, range, or subrange therebetween.

In some embodiments, hydrogen gas (H₂) is also supplied to the reactor for the hydrodechlorination reaction.

In some embodiments, the second method further includes hydrodechlorinating the CF₃CF₂CCl═CHCF₂CF₃ with hydrogen (H₂) in a vapor phase in the presence of a third catalyst to form HFO-153-10mczz.

In some embodiments, the third catalyst includes a nickel-containing catalyst, an iridium/carbon catalyst, a bimetallic catalyst on a carbon support, a gold catalyst on a carbon or other support, a platinum catalyst on a carbon or other support, a palladium catalyst on a carbon or other support, and/or a copper catalyst on a carbon or other support.

In some embodiments, the second method further includes purifying the HFO-153-10mczz. In some embodiments, the purifying included purifying the E-HFO-153-10mczz.

In some embodiments, a third method includes chlorinating a molecule of formula (2), CF₃CF₂CHₙCl₃₋ₙ, in the vapor phase in a reactor to form the CFC-215cb, where n is 1, 2, or 3. In some embodiments, the reactor is a metal alloy reactor. In some embodiments, the metal alloy reactor is an Inconel® tube. In some embodiments, the chlorinating occurs in the absence of a catalyst.

In some embodiments, a composition is formed by any of the foregoing methods.

The HFO-153-10mczz may be isolated and optionally purified prior to use. Suitable uses of HFO-153-10mczz may include, but are not limited to, working fluids in systems utilizing a thermodynamic cycle, a cooling medium, a specialty fluid for thermal management, an immersion cooling fluid, a reactive intermediate, a refrigerant, a heat transfer fluid with or without phase change, a carrier fluid, or a solvent.

The good dielectric properties and suitable boiling point of E-HFO-153-10mczz make it a potential candidate for use as a cooling medium for a lithium-ion battery (LiB) in an automobile.

In other exemplary embodiments, the properties of E-HFO-153-10mczz yield benefits in carrier fluid applications. E-HFO-153-10mczz exhibits good characteristics to enable it to provide traditional carrier fluid behavior for the deposition or removal of soluble compounds, where it readily dissolves, transports, and/or deposits specified media.

In other exemplary embodiments, E-HFO-153-10mczz is used as a solvent for any of a number of various applications. For example, the properties of E-HFO-153-10mczz may yield benefits in solvent cleaning applications. Additional solvent-based of applications for E-HFO-153-10mczz include as a fluid for removal of particulates, greases, oils, and contamination. E-HFO-153-10mczz may also be used as solvents in various applications such as for cleaning (vapor degreasing, flux removal).

In exemplary embodiments, E-HFO-153-10mczz serves as a specialty fluid for thermal management, with slightly elevated boiling temperature ranges, where the product is environmentally friendly (low GWP and ODP), non-flammable, non-conductive, and has low liquid viscosities.

E-HFO-153-10mczz may also be used as a working fluid for immersion cooling, which may be two-phase immersion cooling or single-phase immersion cooling.

Two-phase immersion cooling is an emerging cooling technology for the high-performance cooling market as applied to high performance server systems. It relies on the heat absorbed in the process of vaporizing a liquid immersion cooler fluid to a gas. The fluids used in this application must meet certain requirements to be viable in use. For example, the boiling temperature of the fluid should be in the range between 30-75° C. Generally, this range accommodates maintaining the server components at a sufficiently cool temperature while allowing generated heat to be dissipated sufficiently to an external heat sink. Alternatively, the operating temperature of the server, and the immersion cooling system could be raised or lowered, by using an enclosed system and raising or lowering the pressure within the system to raise or lower the boiling point of a given fluid.

Single phase immersion cooling has a long history in computer server cooling. There is no phase change in single phase immersion cooling. Instead, the liquid warms as it circulates through the computer server and or heat exchanger, and then is circulated with a pump to a heat exchanger for cooling prior to returning to the server, thus transferring heat away from the computer server. Fluids used for single phase immersion cooling have the same requirements as those for two-phase immersion cooling, except that the boiling temperatures are typically higher than 30-75° C., to reduce loss by evaporation.

In exemplary embodiments, E-HFO-153-10mczz serves as an immersion cooling fluid having an operating temperature range near ambient temperatures. Embodiments of the present disclosure, for example, in comparison to concepts failing to include one or more of the features disclosed herein, provide an immersion cooling fluid for thermal management which is environmentally friendly (i.e., have a low global warming potential (GWP) and zero ozone depletion potential (ODP)).

In exemplary embodiments, the immersion cooling fluid cools a heat generating component by at least partially immersing the heat generating component of a device into the immersion cooling fluid in a liquid state such that heat is transferred from the heat generating component using the immersion cooling fluid. Such devices may include, but are not limited to, high-capacity energy storage devices, electrical components, mechanical components and optical components. Appropriate devices may include, but are not limited to, microprocessors, wafers used to manufacture semiconductor devices, power control semiconductors, electrical distribution switch gear, power transformers, circuit boards, multi-chip modules, packaged and unpackaged semiconductor devices, laser, fuel cells, electrochemical cells and energy storage devices such as batteries.

The opportunities for E-HFO-153-10mczz as a new working fluid exist potentially in a variety of heat transfer applications, including, but not limited to, cooling power electronics, such as, for example, televisions, cell phones, monitors, drones, and avionics devices; battery thermal management in both automotive and stationary systems; powertrains for electronic vehicles; insulated-gate bipolar transistors (IGBTs); electronic devices-data center servers; computer server systems; telecommunication infrastructure; 5G network; displays; military electronics; high temperature mechanical vapor compression heat pumps (HTHPs); stationary air conditioning and chillers, Organic Rankine Cycles (ORCs); and anywhere a working fluid provides a medium to transport heat or in applications where passive evaporative cooling exists, such as, for example, heat pipes.

E-HFO-153-10mczz may be used in numerous applications for the transfer of heat, such as, heat transfer fluids or refrigerants. In one embodiment, E-HFO-153-10mczz may be used to transfer heat from an article. The article may be contacted with a heat transfer media including E-HFO-153-10mczz.

E-HFO-153-10mczz and/or Z—HFO-153-10mczz may be used in various applications including as working fluids. Working fluids provide the medium to transport heat or produce power by mechanical means by expansion. Working fluids are typically in the liquid state at a first region. The working fluid absorbs heat in the first region, vaporizes, and migrates to a second region, having a lower temperature, where it condenses. The working fluid is typically returned to the first region after condensation allowing the heat transfer cycle to be repeated. Working fluids may be used in conjunction with compression, expansion systems, pumps, or in passive evaporative cooling such as heat pipes or thermosyphons.

During use, the working fluid in a first region is exposed to an elevated (first) temperature causing the working fluid to vaporize, thus absorbing thermal energy. The vaporized working fluid migrates to a second region, which is at a lower (second) temperature than the first region. The working fluid condenses in the second region, releasing the thermal energy, which is transported external to the system. The working fluid is subsequently returned to the first region. The working fluid typically cyclically moves between the first region and the second region, transporting thermal energy between the first region and the second region.

Working fluids are selected to undergo a phase transition from the liquid to the gaseous state over the desired operational temperature range of a system, such as a heat pipe or thermosyphon. In some embodiments, the composition of the working fluids includes E-HFO-153-10mczz and/or Z—HFO-153-10mczz. In some embodiments, the operational temperature is at least 0° C., at least 10° C., at least 20° C., at least 30° C., at least 40° C., at least 50° C., at least 60° C., at least 70° C., at least 80° C., at least 90° C., at least 100° C., less than 125° C., less than 120° C., less than 110° C., less than 100° C., less than 90° C., less than 75° C., less than 70° C., less than 65° C., less than 60° C., less than 55° C., and combinations thereof.

The ability of the working fluid to transport heat is related to the heat of vaporization of the working fluids. The greater the heat of vaporization of the working fluids the greater amount of energy that the working fluid will absorb during vaporization and transport across the heat pipe to be released during condensation. In some embodiments, E-HFO-153-10mczz may exhibit a heat of vaporization of at least 35 kilojoules per mole (kJ/mol).

Working fluids may also be selected based at least partially on additional material properties. As the working fluids condense and return to the first region workings fluids having a lower viscosity more easily flow between the regions. In some embodiments, E-HFO-153-10mczz may exhibit a viscosity less than water of the same temperature, over the operational temperature range. In some embodiments, E-HFO-153-10mczz may exhibit a viscosity of less than 0.5 centipoise at 55° C.

E-HFO-153-10mczz as a working fluid for heat transfer applications may be selected based at least partially on the surface tension exhibited by the material. For example, in heat pipe applications, working fluids exhibiting high surface tensions may be more easily transported between the hot region and the cool region. In some embodiments, the selection of the wick materials may enhance the rate at which the condensed working fluid is returned to the hot region of the heat pipe. In some embodiments, the working fluids may exhibit a surface tension less than water of the same temperature, over the operational temperature range. In some embodiments, E-HFO-153-10mczz may exhibit a surface tension of less than 64.5 dyne/cm at 70° C., less than 66.3 dyne/cm at 60° C., and/or less than 67.9 dyne/cm at 50° C.

The working fluids may also be selected based at least partially on other thermodynamic properties of the materials. Working fluids exhibiting a lower specific heat and/or a lower thermal conductivity than water at the same temperature may enhance energy transport between the hot region and the cool region of a heat pipe. In some embodiments, the working fluids may exhibit a specific heat of less than 4.2 Joules per gram Kelvin degree. In some embodiments, E-HFO-153-10mczz may exhibit a thermal conductivity of less than 0.6 watts per meter Kelvin degree at 20° C.

The working fluids may also be selected to exhibit a dielectric constant suitable for electrical applications. In general, materials exhibiting a low dielectric constant provide increased electrical isolation of the electrical components immersed therein. In some embodiments, the dielectric constant of the working fluids is less than about 8 over the operational frequency range (0 to 20 GHz). Suitable dielectric working fluids include E-HFO-153-10mczz having a dielectric constant over the operational frequency range (0 to 20 GHz) of less than 7.3, or less than 5.5, or less than 5.0, or less than 4.0, or less than 3.5, or less than 2.7, or less than 2.5, or less than 2.0, or less than 1.9, or less than 1.8, or less than 1.5. Other embodiments include compounds and mixtures having a dielectric constant greater than 1.0 and less than 8.0 or greater than 2.0 and less than 7.3 or greater than 2.5 and less than 5.5 or greater than 3.5 and less than 5.0.

Table 1 shows certain properties relevant for working fluids for HFO-10mczz compared to other similar compounds.

TABLE 1

| | method = | | | |
|---|---|---|---|---|
| | basis | | liquid | |
| | set = | MP2 | at | |
| | dipole | 6-311G (d, p) | 25° C. | |
| | moment | polarizability | density | dielectric |
| Fluid | (Debye) | (a₀³) | (g/cm³) | constant |
| E-C₂F₅CF═CFC₂F₅ | 0.00 | 63.27 | 1.64 | 1.75* |
| (E-HFO-151-12mcyy) | | | | |
| E-C₂F₅CH═CHC₂F₅ | 0.10 | 62.74 | 1.52 | 1.82* |
| (E-HFO-153-10mczz) | | | | |
| C₃F₇CH═CHCF₃ | 0.24 | 62.81 | 1.51 | 2.09* |
| (HFO-153-10mzz) | | | | |
| (CF₃)₂CFCH═CHCF₃ | 0.20 | 62.66 | 1.49 | 1.85* |
| (HFO-153-10mzzy) | | | | |
| Z-C₂F₅CF═CFC₂F₅ | 0.43 | 63.45 | 1.64 | 1.59** |
| (Z-FO-151-12mcyZ) | | | | |
| CF₃CH═CHCF₂CF₂Cl | 0.59 | 60.85 | 1.49 | 1.74** |
| CF₃CH═CHCF₂CF₂CF₂Cl | 0.84 | 71.90 | 1.53 | 1.94** |
| CF₃CH═CHCF₂CFClCF₃ | 0.59 | 72.14 | 1.74 | 2.09** |
| CF₃CH═CHCFClCF₃ | 0.68 | 61.66 | 1.57 | 2.28** |
| C₄F₉CH═CFH | 1.48 | 63.26 | 1.55 | 3.77** |
| (HFO-153-10ze) | | | | |
| E-CF₃CCl═CFCl | 0.98 | 50.72 | 1.53 | 2.67** |
| (E-CFO-1214xbE) | | | | |
| CF₃CF═CCl₂ | 1.26 | 50.90 | 1.53 | 3.42** |
| (CFO-1214ya) | | | | |
| C₂F₅CH═CFC₂F₅ | 1.41 | 62.88 | 1.61 | 3.46** |
| (HFO-152-11mcyz) | | | | |
| BrCF₂CF₂CH═CH₂ | 2.16 | 57.52 | 1.73 | 4.81** |
| Z-CF₃CCl═CFCl | 1.78 | 50.30 | 1.53 | 5.45** |
| (Z-CFO-1214xbZ) | | | | |
| Z-HCF₂CF═CHCl | 1.74 | 41.37 | 1.32 | 7.28** |
| (Z-HCFO-1233ydZ) | | | | |

*measured using ASTM D924
**calculated using conventional methods known in the art

Additional additives may be added to the working fluid. Suitable additives include linear hydrocarbons, linear halocarbons, cyclic hydrocarbons, cyclic halocarbons, heptafluorocyclopentane, alcohols (e.g., methanol, ethanol, isopropanol), ethers, halogenated ethers, ketones, and halogenated ketones. Examples of suitable additives include pentane (bp 36° C.), hexane (bp 69° C.), heptane (bp 98° C.), octane (bp 125° C.), cyclopentane (bp 49° C.), cyclohexane (bp 80° C.), cycloheptane (bp 118° C.), methyl cyclobutane (bp 39° C.), methyl cyclopentane (bp 72° C.), diethyl ether (bp 35° C.), diisopropyl ether (bp 69° C.), C₄F₉OCH₃ (CAS 163702-07-6), C₄F₉OCH₂CH₃(CAS 163702-05-4); i-C₄F₉OCH₂CH₃(CAS 163702-06-5), (CF₃)₂CFCF(OCH₃)CF₂CF₃ (73DE, CAS 132182-92-4), C₃F₇OCH₃ (CAS 375-03-1), (CF₃)₂CFCF(OCH₂CH₃)CF₂CF₂CF₃ (HFE 7500, CAS 297730-93-9), 1,1,1,2,3,3-hexafluoro-4-(1,1,2,3,3,3-hexafluoropropoxy)pentane (HFE 7600, CAS 870778-34-0), Furan, 2,3,3,4,4-pentafluorotetrahydro-5-methoxy-2,5-bis[1,2,2,2-tetrafluoro-1-(trifluoromethyl)ethyl]-(HFE 7700), [CAS 812-05-4], and 1,1,1,2,4,4,5,5,5-nonafluoro-(2-trifluoromethyl)-3-pentanone (Novec 1230, CAS 756-13-8).

### EXAMPLES

Exemplary examples of methods in the formation of HFO-153-10mczz are shown below.

### Example 1

Coupling HCFC-225Ca with Copper in DMF

100 g (0.49 mol) of 3,3-dichloro-1,1,1,2,2-pentafluoropropane (HCFC-225ca), 8 g (1.28 mol) of pure metallic copper (Cu) powder, 6 g (0.013 mol) of 2,2-bipyridine, and 200 g of dimethylformamide (DMF) were charged into a 400-mL Hastelloy® C shaker tube. The starting HCFC-225ca contained about 2.2% of HCFC-225cb. The reactor was chilled to 0° C., then a vacuum was pulled. The reaction mixture was then heated to 90° C. with agitation and agitated at 90° C. for 4 hours. The pressure of the reactor increased to 21 psig at 90° C. The pressure then dropped to −5 psig after the reactor was cooled down to room temperature.

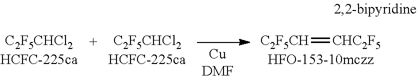

The liquid phase of the reaction was analyzed by GC-FID-MS. The data is reported in Table 2 by area percent by gas chromatography flame ionization detection (GC-FID). The selectivity to E-HFO-153-10mczz was about 85%.

TABLE 2

| Compound | GC-FID area % |
|---|---|
| C₅H₂F6 | 0.1356 |
| E-C₂F₅CH═CHC₂F₅ (E-HFO-153-10mczz) | 10.7282 |
| CF₃CF═CHCl (HCFO-1224yd) | 0.4570 |
| CF₃CF₂CH₂Cl (HCFC-235cb) | 0.1539 |
| C₆H₂F8 | 0.0292 |
| C₆H₂F8 | 0.3383 |
| Z-C₂F₅CH═CHC₂F₅ (Z-HFO-153-10mczz) | 0.5492 |
| C₄H₂ClF₅ (HCFO-1335) | 0.0664 |
| CHF₂CClFCH₂F (HCFC-224ba) | 0.0360 |
| C₄H₂ClF₅ (HCFO-1335) | 0.0352 |
| CClF₂CF₂CHClF (HCFC-225cb) | 0.2790 |
| DMF | 87.0712 |
| Others | 0.1207 |

### Example 2

### Chlorination of HCFC-225Ca/HCFC-225cb Mixture to CFC-215Ca/CFC-215cb

An Inconel 600 tube having dimensions of 15″ length×0.5″ outside diameter (OD) and 0.43″ inside diameter (ID) was installed as a reactor. After an N₂ purge to remove the air from reactor, a mixture of 3,3-dichloro-1,1,1,2,2-pentafluoropropane (HCFC-225ca) and 1,3-dichloro-1,1,2,2,3-pentafluoropropane (HCFC-225cb) and N₂ and Cl₂ gas was fed into the reactor at condition shown in Table 3. The reactor effluent was analyzed by online GC-MS-FID. The reaction was free of a catalyst.

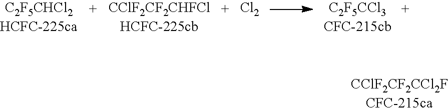

As shown in Table 3, HCFC-225ca and HCFC-225cb were efficiently chlorinated to 1,1,1-trichloropentafluoropropane (CFC-215cb) and 1,1,3-trichloropentafluoropropane (CFC-215ca), respectively, without a catalyst.

TABLE 3

| | | | | | | | | | 225ca | 225cb |
|---|---|---|---|---|---|---|---|---|---|---|
| Time | Mole % | | | T | P | Organic | Cl₂ | N₂ | mol % | mol % |
| (hr) | 225ca | 225cb | 215 | (° C.) | (psi) | (mL/hr) | (sccm) | (sccm) | conversion | conversion |
| 1 | 61.68 | 38.32 | 0.00 | 200 | −1.0 | 0.50 | 4.29 | 4.28 | 0 | 0 |
| 2 | 61.49 | 38.51 | 0.00 | 200 | −1.1 | 0.50 | 4.29 | 4.28 | 0 | 0 |
| 3 | 61.32 | 38.68 | 0.00 | 200 | −1.0 | 0.50 | 4.28 | 4.25 | 0 | 0 |
| 4 | 54.40 | 36.40 | 9.20 | 300 | −1.1 | 0.50 | 13.91 | 4.23 | 11.52 | 5.49 |
| 5 | 54.63 | 36.38 | 8.99 | 300 | −1.0 | 0.50 | 13.90 | 4.26 | 11.15 | 5.54 |
| 6 | 53.7 | 36.02 | 10.21 | 300 | −1.0 | 0.50 | 13.90 | 4.26 | 12.55 | 6.47 |
| 7 | 44.94 | 31.99 | 23.07 | 325 | −0.9 | 0.50 | 13.90 | 4.26 | 26.92 | 16.93 |
| 9 | 42.04 | 30.92 | 27.03 | 325 | −0.9 | 0.50 | 13.91 | 4.25 | 31.62 | 19.71 |
| 11 | 22.08 | 21.06 | 56.86 | 350 | −1.0 | 0.50 | 13.91 | 4.25 | 64.09 | 45.31 |
| 12 | 20.69 | 20.61 | 58.70 | 350 | −1.1 | 0.50 | 13.91 | 4.25 | 66.35 | 46.49 |
| 14 | 30.59 | 24.22 | 45.19 | 375 | 1.7 | 0.50 | 13.91 | 4.25 | 50.25 | 37.10 |

### Example 4

### Chlorination of HFC-245cb to HCFC-225ca and CFC-215cb

An Inconel 600 tube having dimensions of 15″ length×0.5″ OD and 0.43″ ID was installed as a reactor. After an N₂ purge to remove the air from the reactor, a mixture of 1,1,1,2,2-pentafluoropropane (HCFC-245cb) and Cl₂ gas was fed into the reactor at atmospheric pressure and the conditions shown in Table 4 for a chlorination reaction. The reactor effluent was analyzed by online GC-MS-FID. The reaction was free of a catalyst.

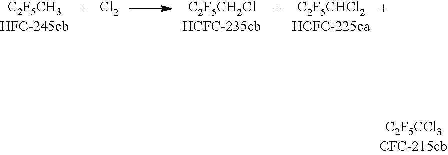

TABLE 4

| | | HFC-245cb | |
|---|---|---|---|
| Time (hr) | T (° C.) | (sccm) | N₂ (sccm) |
| 1 | 375 | 8.00 | 8.00 |
| 2 | 375 | 8.00 | 7.97 |
| 3 | 375 | 8.00 | 7.98 |
| 4 | 375 | 7.35 | 12.08 |
| 5 | 375 | 7.36 | 12.08 |
| 6 | 375 | 7.33 | 12.03 |
| 7 | 375 | 7.36 | 15.04 |
| 8 | 375 | 7.35 | 15.01 |
| 9 | 375 | 7.32 | 15.04 |
| 10 | 400 | 7.99 | 7.98 |
| 11 | 400 | 7.99 | 7.94 |
| 12 | 400 | 7.99 | 7.98 |
| 13 | 400 | 7.36 | 12.07 |
| 14 | 400 | 7.38 | 12.05 |
| 15 | 400 | 7.36 | 12.07 |
| 16 | 400 | 7.36 | 15.05 |
| 17 | 400 | 7.35 | 15.01 |
| 18 | 400 | 7.39 | 15.06 |
| 19 | 399 | 8.06 | 16.08 |
| 20 | 400 | 8.05 | 16.09 |
| 21 | 400 | 8.07 | 16.05 |
| 22 | 400 | 7.97 | 20.07 |
| 23 | 400 | 7.96 | 20.07 |
| 24 | 400 | 7.95 | 20.09 |
| 25 | 425 | 7.96 | 16.08 |
| 26 | 425 | 7.97 | 16.05 |
| 27 | 425 | 7.96 | 16.09 |
| 28 | 425 | 7.95 | 20.09 |
| 29 | 425 | 7.95 | 20.08 |
| 30 | 425 | 7.97 | 20.05 |

As shown in Table 5, HFC-245cb was efficiently chlorinated to 3,3-dichloro-1,1,1,2,2-pentafluoropropane (HCFC-225ca) and 1,1,1-trichloropentafluoropropane (CFC-215cb) without a catalyst.

TABLE 5

| | Reactor Effluent (mol %) | | | | | | |
|---|---|---|---|---|---|---|---|
| Time | HFC- | HCFO- | HCFC- | HCFC- | CFC- | HCFC- | |
| (hr) | 245cb | 1224yd | 235cb | 225ca | 215cb | 224ba | other |
| 1 | 69.30 | 0.05 | 11.15 | 9.21 | 9.91 | 0.01 | 0.36 |
| 2 | 68.90 | 0.05 | 11.17 | 9.30 | 10.21 | 0.02 | 0.35 |
| 3 | 70.17 | 0.05 | 11.11 | 8.72 | 9.34 | 0.13 | 0.48 |
| 4 | 65.49 | 0.04 | 10.98 | 9.66 | 12.84 | 0.20 | 0.79 |
| 5 | 65.31 | 0.04 | 10.94 | 9.62 | 13.07 | 0.21 | 0.80 |
| 6 | 65.00 | 0.04 | 10.94 | 9.69 | 13.29 | 0.21 | 0.82 |
| 7 | 65.06 | 0.04 | 10.98 | 9.79 | 13.05 | 0.21 | 0.87 |
| 8 | 65.22 | 0.04 | 10.98 | 9.75 | 12.93 | 0.23 | 0.85 |
| 9 | 65.33 | 0.04 | 11.00 | 9.73 | 12.84 | 0.22 | 0.85 |
| 10 | 59.13 | 0.07 | 10.31 | 11.21 | 18.57 | 0.00 | 0.71 |
| 11 | 57.59 | 0.07 | 10.33 | 11.62 | 20.17 | 0.00 | 0.22 |
| 12 | 60.43 | 0.07 | 10.52 | 11.26 | 17.48 | 0.00 | 0.23 |
| 13 | 42.68 | 0.05 | 8.14 | 10.88 | 36.87 | 0.00 | 1.37 |
| 14 | 42.44 | 0.05 | 8.26 | 11.06 | 37.72 | 0.00 | 0.47 |
| 15 | 43.62 | 0.05 | 8.43 | 11.05 | 35.56 | 0.00 | 1.29 |
| 16 | 39.52 | 0.05 | 7.99 | 10.85 | 40.65 | 0.00 | 0.94 |
| 17 | 39.47 | 0.05 | 8.04 | 10.90 | 39.39 | 0.50 | 1.65 |
| 18 | 40.40 | 0.05 | 8.20 | 11.00 | 38.33 | 0.50 | 1.52 |
| 19 | 40.84 | 0.05 | 8.10 | 11.03 | 39.19 | 0.00 | 0.79 |
| 20 | 41.61 | 0.05 | 8.43 | 11.32 | 38.35 | 0.13 | 0.12 |
| 21 | 41.15 | 0.05 | 8.41 | 11.23 | 37.57 | 0.40 | 1.19 |
| 22 | 40.49 | 0.05 | 8.45 | 11.40 | 39.32 | 0.15 | 0.14 |
| 23 | 40.75 | 0.04 | 8.53 | 11.47 | 38.92 | 0.15 | 0.13 |
| 24 | 41.46 | 0.04 | 8.68 | 11.53 | 37.99 | 0.15 | 0.14 |
| 25 | 33.16 | 0.17 | 6.38 | 11.95 | 45.99 | 0.64 | 1.70 |
| 26 | 31.84 | 0.19 | 6.13 | 11.55 | 48.10 | 0.60 | 1.60 |
| 27 | 33.92 | 0.20 | 6.70 | 11.84 | 45.23 | 0.57 | 1.54 |
| 28 | 22.06 | 0.13 | 3.81 | 7.89 | 63.34 | 0.00 | 2.77 |
| 29 | 23.19 | 0.15 | 4.37 | 8.68 | 60.88 | 0.00 | 2.74 |
| 30 | 22.58 | 0.14 | 4.31 | 8.53 | 61.62 | 0.00 | 2.82 |

### Example 5

Coupling CFC-215cb with Ru/SiC Catalyst

10 mL of 2% Ru/SiC catalyst pellets was loaded into a 12-inch long, half-inch OD Monel 400 reactor. After an N₂ purge to remove the air from reactor, the catalyst was activated by H₂ at 150° C. for 2 hours. CFC-215cb and H₂ were then fed into the reactor at a temperature of 130° C., a pressure of 150 psi, an organic flowrate of 3.57 cc/hr, an N₂ flowrate of 30.4 standard cubic centimeters per minute (sccm), and an H₂ flowrate of 30.4 sccm.

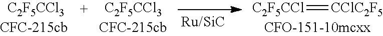

The reactor effluent was analyzed by online GC-MS-FID. As shown in Table 6 and Table 7, CFC-215cb reached high conversion and HFO-151-10mcxx was produced at high selectivity.

TABLE 6

| | Reactor Effluent (mol %) | | | | | | | | |
|---|---|---|---|---|---|---|---|---|---|
| Time | | | | | | | | E-151- | Z151- |
| (hr) | Other | 245cb | 1215xc | 225ca | 215aa | 215cb | 215ca | 10mcxx | 10mcxx |
| 2 | 3.53 | 2.14 | 1.50 | 5.68 | 1.05 | 0.00 | 3.85% | 71.07% | 11.19% |
| 10 | 0.10 | 1.65 | 1.15 | 5.23 | 1.44 | 0.00 | 4.02% | 74.68% | 11.73% |
| 20 | 0.00 | 1.58 | 1.06 | 4.78 | 1.60 | 0.40 | 4.12% | 74.73% | 11.73% |
| 30 | 0.00 | 1.70 | 1.28 | 4.73 | 1.35 | 0.00 | 4.09% | 74.97% | 11.88% |
| 40 | 0.00 | 1.57 | 1.15 | 4.62 | 1.45 | 0.00 | 4.06% | 75.05% | 12.10% |
| 50 | 0.00 | 1.37 | 0.37 | 5.35 | 0.51 | 0.00 | 3.80% | 76.25% | 12.35% |
| 60 | 0.00 | 1.43 | 0.38 | 5.55 | 0.50 | 0.00 | 3.91% | 76.12% | 12.10% |
| 70 | 0.00 | 1.15 | 0.30 | 4.92 | 0.55 | 0.46 | 3.75% | 76.40% | 12.47% |
| 80 | 0.00 | 1.25 | 0.33 | 4.93 | 0.54 | 0.37 | 3.89% | 76.40% | 12.30% |
| 90 | 0.00 | 1.13 | 0.29 | 4.83 | 0.58 | 0.52 | 3.91% | 76.32% | 12.42% |
| 100 | 0.00 | 1.15 | 0.30 | 4.73 | 0.59 | 0.47 | 3.95% | 76.51% | 12.30% |

TABLE 7

| | 215cb | 151-10mcxx |
|---|---|---|
| Time (hr) | conversion (mol %) | selectivity (mol %) |
| 2 | 100.0% | 85.3% |
| 10 | 100.0% | 86.5% |
| 20 | 99.6% | 86.8% |
| 30 | 100.0% | 86.9% |
| 40 | 100.0% | 87.2% |
| 50 | 100.0% | 88.6% |
| 60 | 100.0% | 88.2% |
| 70 | 99.5% | 89.3% |
| 80 | 99.6% | 89.0% |
| 90 | 99.5% | 89.2% |
| 100 | 99.5% | 89.2% |

### Example 6

Hydrodechlorination of CFO-151-10mcxx with 56% Ni Catalyst

10 mL of a 56% nickel-containing ⅛″ tablet catalyst (BASF E474 TR, BASF, Ludwigshafen, Germany) was loaded into a 12-inch long, half-inch OD Monel 400 reactor. After an N₂ purge to remove air from reactor, the catalyst was activated by H₂ at 250° C. for 8 hours. CFO-151-10mcxx and H₂ were then feed into the reactor at a temperature of 250° C., a pressure of 150 psi, an organic flowrate of 0.75 cc/hr, an N₂ flowrate of 25.1 sccm, and an H₂ flowrate of 25.1 sccm.

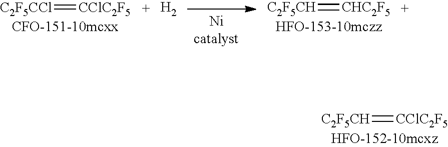

The reactor effluent was analyzed by online GC-MS-FID. As shown in Table 8 and Table 9 below, CFO-151-10mcxx reached high conversion and HFO-153-10mczz was produced at good selectivity.

TABLE 8

| | Reactor Effluent (mol %) | | | | | | | | |
|---|---|---|---|---|---|---|---|---|---|
| Time | | E-153- | 55- | | Z-153- | E-152- | | E-151- | Z-151- |
| (hr) | Other | 10mczz | 10mcff | 1549 | 10mczz | 10mcxz | C₆H₇F₅ | 10mcxx | 10mcxx |
| 3 | 1.86 | 8.75 | 4.14 | 5.80 | 53.17 | 18.83 | 5.69 | 0.00 | 0.00 |
| 4 | 1.87 | 8.24 | 3.13 | 5.40 | 54.40 | 18.07 | 5.71 | 1.34 | 0.00 |
| 5 | 1.76 | 8.07 | 2.92 | 5.38 | 55.04 | 17.84 | 5.50 | 1.73 | 0.00 |
| 6 | 2.03 | 8.47 | 3.27 | 5.48 | 54.69 | 16.29 | 6.33 | 1.45 | 0.00 |
| 7 | 1.99 | 8.48 | 3.30 | 5.49 | 54.97 | 15.73 | 6.58 | 1.54 | 0.00 |
| 8 | 1.59 | 8.13 | 2.83 | 5.46 | 55.81 | 17.03 | 5.39 | 2.19 | 0.00 |
| 9 | 1.41 | 8.01 | 2.64 | 5.45 | 56.23 | 17.44 | 4.77 | 2.65 | 0.00 |
| 10 | 1.22 | 7.71 | 2.42 | 5.34 | 56.77 | 18.06 | 4.20 | 3.08 | 0.00 |
| 11 | 1.08 | 7.51 | 2.25 | 5.26 | 57.03 | 18.31 | 3.75 | 3.78 | 0.00 |
| 12 | 1.00 | 7.43 | 2.15 | 5.24 | 57.20 | 18.38 | 3.48 | 4.17 | 0.00 |
| 13 | 0.97 | 7.46 | 2.14 | 5.30 | 57.90 | 18.39 | 3.38 | 4.45 | 0.00 |
| 14 | 0.93 | 7.33 | 2.05 | 5.28 | 58.05 | 18.32 | 3.20 | 4.85 | 0.00 |
| 15 | 0.87 | 7.24 | 1.98 | 5.22 | 57.91 | 18.35 | 3.00 | 5.43 | 0.00 |
| 17 | 0.61 | 6.48 | 1.50 | 4.70 | 57.04 | 18.74 | 2.00 | 8.93 | 0.00 |
| 18 | 0.73 | 6.81 | 1.70 | 4.96 | 57.61 | 17.87 | 2.47 | 7.85 | 0.00 |

TABLE 9

| | 151-10mcxx | 152-10mcxz | 153-10mczz |
|---|---|---|---|
| Time (hr) | conversion (%) | selectivity (%) | selectivity (%) |
| 3 | 100.0 | 19.2 | 63.1 |
| 4 | 98.7 | 18.4 | 63.8 |
| 5 | 98.3 | 18.2 | 64.2 |
| 6 | 98.6 | 16.6 | 64.5 |
| 7 | 98.5 | 16.1 | 64.7 |
| 8 | 97.8 | 17.3 | 65.0 |
| 9 | 97.3 | 17.7 | 65.2 |
| 10 | 96.9 | 18.3 | 65.3 |
| 11 | 96.2 | 18.5 | 65.2 |
| 12 | 95.8 | 18.6 | 65.3 |
| 13 | 95.5 | 18.6 | 66.0 |
| 14 | 95.1 | 18.5 | 66.0 |
| 15 | 94.6 | 18.5 | 65.7 |
| 17 | 91.1 | 18.9 | 63.9 |
| 18 | 92.2 | 18.0 | 64.9 |

### Example 7

Hydrodechlorination of CFO-151-10mcxx with 1% Ir/C Catalyst

10 mL of a 1% Ir/C catalyst was loaded into a 12-inch long, half-inch OD Monel 400 reactor. After an N₂ purge to remove air from the reactor, the catalyst was activated by H₂ at 250° C. for 8 hours. CFO-151-10mcxx and H₂ were then feed into the reactor at a pressure of 150 psi, an organic flowrate of 0.86 cc/hr, an H₂ flowrate of 17.9 sccm, and the temperature indicated in Table 11.

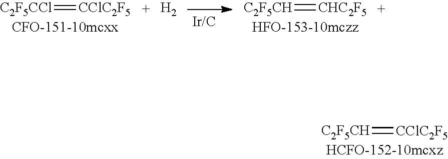

The reactor effluent was analyzed by online GC-MS-FID. As shown in Table 10 and Table 11, CFO-151-10mcxx reached high conversion and HFO-153-10mczz was produced. PFH in Table 10 is perfluorohexyne.

TABLE 10

| | Reactor Effluent (mol %) | | | | | | | | |
|---|---|---|---|---|---|---|---|---|---|
| Time | | | E-153- | 55- | Z-153- | E-152- | Z-152- | E-151- | Z-151- |
| (hr) | Other | PFH | 10mczz | 10mcff | 10mczz | 10mcxz | 10mcxz | 10mcxx | 10mcxx |
| 6 | 0.00 | 12.55 | 0.00 | 7.64 | 3.79 | 0.00 | 0.00 | 76.03 | 0.00 |
| 20 | 0.00 | 11.15 | 0.00 | 1.01 | 3.80 | 0.00 | 0.00 | 75.88 | 8.16 |
| 21 | 0.32 | 8.70 | 0.27 | 0.43 | 2.32 | 1.03 | 0.00 | 80.55 | 6.38 |
| 24 | 0.00 | 8.72 | 0.00 | 0.93 | 2.44 | 1.23 | 0.00 | 79.10 | 7.58 |
| 25 | 0.00 | 4.47 | 1.33 | 2.29 | 0.88 | 30.27 | 0.62 | 53.20 | 6.51 |
| 26 | 0.27 | 1.75 | 1.30 | 1.65 | 0.71 | 49.86 | 1.31 | 37.39 | 4.77 |
| 27 | 0.77 | 1.66 | 1.74 | 2.14 | 0.93 | 50.68 | 1.73 | 34.60 | 4.61 |
| 28 | 0.97 | 1.49 | 2.14 | 2.84 | 1.09 | 49.88 | 1.84 | 33.68 | 4.82 |
| 35 | 3.77 | 1.43 | 5.21 | 9.00 | 2.10 | 56.84 | 0.36 | 14.32 | 5.03 |
| 36 | 3.82 | 0.72 | 6.45 | 10.92 | 2.65 | 63.27 | 0.41 | 7.93 | 1.67 |
| 39 | 2.27 | 0.70 | 6.52 | 11.17 | 3.08 | 56.95 | 1.85 | 12.98 | 2.69 |
| 40 | 0.92 | 2.79 | 1.31 | 9.04 | 4.85 | 55.78 | 1.91 | 20.49 | 1.82 |

TABLE 11

| | | 151-10mcxx | | |
|---|---|---|---|---|
| | | conversion | 152-10mcxz | 153-10mczz |
| Time (hr) | T (° C.) | (%) | selectivity (%) | selectivity (%) |
| 6 | 70 | 24.0 | 0.0 | 15.8 |
| 20 | 70 | 16.0 | 0.0 | 23.8 |
| 21 | 124 | 13.1 | 8.0 | 20.4 |
| 24 | 124 | 13.3 | 9.2 | 18.3 |
| 25 | 200 | 40.3 | 76.7 | 5.5 |
| 26 | 200 | 57.8 | 88.9 | 3.5 |
| 27 | 201 | 60.8 | 87.3 | 4.4 |
| 28 | 201 | 61.5 | 85.5 | 5.3 |
| 35 | 199 | 80.6 | 74.4 | 9.5 |
| 36 | 199 | 90.4 | 73.5 | 10.5 |
| 39 | 201 | 84.3 | 71.6 | 11.7 |
| 40 | 125 | 77.7 | 75.1 | 8.0 |

### Example 8

Hydrodechlorination of CFO-151-10mcxx with Bimetal Catalyst

6 mL of a bimetal catalyst on a solid carbon support was loaded into a 12-inch long, half-inch OD Monel 400 reactor. After an N₂ purge to remove air from the reactor, the catalyst was activated by H₂ at 250° C. for 8 hours. CFO-151-10mcxx and H₂ were then feed into the reactor at a pressure of 150 psi, an organic flowrate of 3.20 cc/hr, an H₂ flowrate of 25.0 sccm, and the temperature and N₂ flowrate indicated in Table 13.

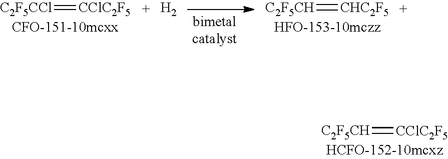

The reactor effluent was analyzed by online GC-MS-FID. As shown in Table 12 and Table 13, CFO-151-10mcxx reached high conversion and HFO-153-10mczz was produced with high selectivity.

TABLE 12

| | Reactor Effluent (mol %) | | | | |
|---|---|---|---|---|---|
| | E-152- | Z-152- | E-151- | Z-151- | |
| Time (hr) | 10mcxz | 10mcxz | 10mcxx | 10mcxx | others |
| 1 | 17.51 | 0.95 | 69.07 | 8.10 | 4.37 |
| 2 | 39.07 | 4.70 | 43.59 | 7.45 | 4.89 |
| 3 | 41.31 | 4.55 | 43.05 | 6.94 | 4.14 |
| 4 | 40.62 | 4.52 | 44.34 | 6.83 | 3.69 |
| 5 | 41.06 | 4.45 | 44.27 | 6.71 | 3.52 |
| 6 | 36.84 | 4.14 | 48.69 | 7.38 | 2.95 |
| 7 | 65.62 | 4.80 | 14.42 | 2.02 | 12.45 |
| 8 | 66.26 | 4.72 | 7.60 | 1.07 | 19.41 |
| 9 | 67.46 | 4.82 | 8.24 | 1.10 | 17.57 |
| 10 | 68.50 | 5.14 | 9.19 | 1.21 | 15.24 |
| 11 | 67.36 | 5.17 | 11.77 | 1.57 | 13.43 |
| 12 | 71.32 | 5.55 | 9.64 | 1.11 | 11.74 |
| 13 | 73.88 | 6.04 | 7.53 | 0.68 | 11.22 |
| 14 | 75.18 | 6.52 | 6.40 | 0.50 | 10.59 |
| 15 | 76.54 | 6.30 | 5.85 | 0.45 | 10.23 |
| 16 | 76.26 | 6.34 | 6.16 | 0.46 | 10.16 |
| 17 | 76.44 | 6.34 | 6.00 | 0.45 | 10.17 |
| 18 | 76.65 | 6.39 | 5.91 | 0.43 | 9.97 |
| 19 | 10.02 | 76.83 | 6.29 | 5.82 | 10.02 |
| 20 | 8.90 | 76.30 | 6.18 | 7.46 | 8.90 |
| 21 | 9.50 | 74.75 | 6.17 | 8.31 | 9.50 |

TABLE 13

| Time | | | 151-10mcxx | 152-10mcxz |
|---|---|---|---|---|
| (hr) | T (° C.) | N₂ (sccm) | conversion (%) | selectivity (%) |
| 1 | 150 | 25.05 | 22.8 | 82.9 |
| 2 | 200 | 25.06 | 49.0 | 89.4 |
| 3 | 200 | 25.06 | 50.0 | 91.7 |
| 4 | 200 | 25.06 | 48.8 | 92.4 |
| 5 | 200 | 25.06 | 49.0 | 92.8 |
| 6 | 200 | 25.08 | 43.9 | 93.3 |
| 7 | 230 | 10.21 | 83.6 | 86.0 |
| 8 | 230 | 0.20 | 91.3 | 81.0 |
| 9 | 230 | 0.24 | 90.7 | 83.4 |
| 10 | 230 | 0.17 | 89.6 | 85.6 |
| 11 | 230 | 0.18 | 86.7 | 86.9 |
| 12 | 230 | 0.19 | 89.2 | 88.6 |
| 13 | 230 | 0.23 | 91.8 | 89.2 |
| 14 | 230 | 0.18 | 93.1 | 89.7 |
| 15 | 230 | 0.20 | 93.7 | 90.3 |
| 16 | 230 | 0.19 | 93.4 | 90.4 |
| 17 | 230 | 0.21 | 93.6 | 90.3 |
| 18 | 230 | 0.24 | 93.7 | 90.4 |
| 19 | 230 | 0.18 | 93.7 | 90.4 |
| 20 | 230 | 0.21 | 91.9 | 91.1 |
| 21 | 230 | 0.19 | 91.0 | 90.4 |

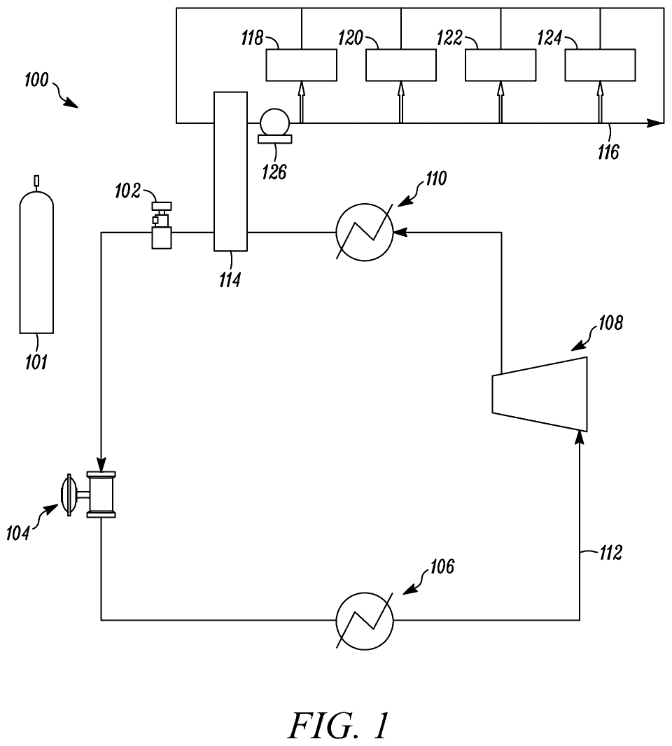

FIG. 1 illustrates a system 100 having at least a first loop 112 and an optional second loop 116. A supply 101 of one of E-C₂F₅CF═CFC₂F₅(E-HFO-151-12mcyy), E-C₂F₅CH═CHC₂F₅, (E-HFO-153-10mczz), C₃F₇CH═CHCF₃ (HFO-153-10mzz), (CF₃)₂CFCH═CHCF₃ (HFO-153-10mzzy), Z—C₂F₅CF═CFC₂F₅ (z-FO-151-12mcyZ) or a one of E-C₂F₅CF═CFC₂F₅(E-HFO-151-12mcyy), E-C₂F₅CH═CHC₂F₅, (E-HFO-153-10mczz), C₃F₇CH═CHCF₃ (HFO-153-10mzz), (CF₃)₂CFCH═CHCF₃ (HFO-153-10mzzy), Z—C₂F₅CF═CFC₂F₅ (z-FO-151-12mcyZ) is available for charging loop system 112 or for recharging or replacing existing refrigerants in loop system 11. Loop system 112 generally includes at least one exchanger 110, a pressure regulator 104, at least one exchanger 106, a compressor 108, and charging valve 102 that may be arranged in seriatim as shown.

FIG. 1 also illustrates an optional second loop 116 having a third exchanger that exchanges thermal energy between loops 112 and 116. A motive force 126 (compressor, pump, etc.) which circulates E-C₂F₅CF═CFC₂F₅(E-HFO-151-12mcyy), E-C₂F₅CH═CHC₂F₅, (E-HFO-153-10mczz), C₃F₇CH═CHCF₃ (HFO-153-10mzz), (CF₃)₂CFCH═CHCF₃ (HFO-153-10mzzy), Z—C₂F₅CF═CFC₂F₅ (z-FO-151-12mcyZ) or blends thereof to various electrical components, such as batteries, electric motors, computer components in need of thermal management to regulate temperature. Thermal energy from one or more of components 118, 120, 122 and 124 is used to exchange heat with the working fluid and can be used to heat other fluids, e.g., air, water or other fluid. Valving (not shown) is included in all flow lines to assist with the thermal management, as are thermal energy exchangers upstream or downstream of components 118, 120, 122 and 124. Although four components in need of thermal management are illustrated, less components or more components can be included.

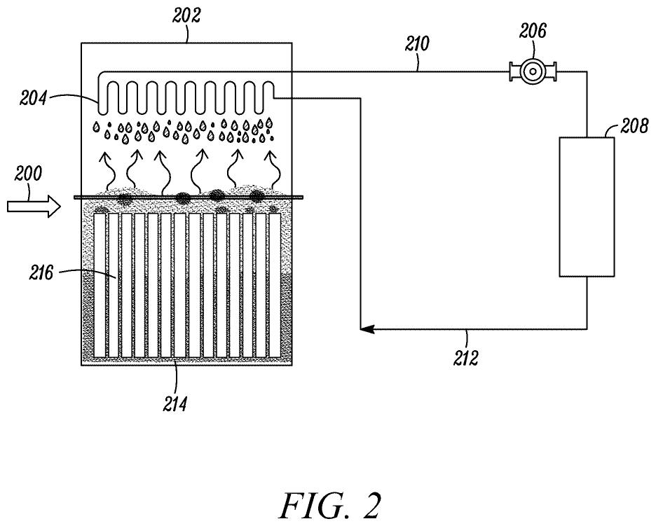

FIG. 2 illustrates an exemplary immersion cooling embodiment 200 including a two-phase tank 202, one or more condenser units, pump 206, a liquid chiller unit, a condenser return line 210 and chilled fluid feed line 212, and immersion fluid 214 and an electric component(s) 216. The immersion fluid comprises, consists essentially of, or consists of one or more of E-C₂F₅CF═CFC₂F₅(E-HFO-151-12mcyy), E-C₂F₅CH═CHC₂F₅, (E-HFO-153-10mczz), C₃F₇CH═CHCF₃ (HFO-153-10mzz), (CF₃)₂CFCH═CHCF₃ (HFO-153-10mzzy), Z—C₂F₅CF═CFC₂F₅ (z-FO-151-12mcyZ) or a one of E-C₂F₅CF═CFC₂F₅(E-HFO-151-12mcyy), E-C₂F₅CH═CHC₂F₅, (E-HFO-153-10mczz), C₃F₇CH═CHCF₃ (HFO-153-10mzz), (CF₃)₂CFCH═CHCF₃ (HFO-153-10mzzy), Z—C₂F₅CF═CFC₂F₅ (z-FO-151-12mcyZ)

The thermal regulating fluid described here comprises, consists essentially of, or consists of one or more of E-C₂F₅CF═CFC₂F₅ (E-HFO-151-12mcyy), E-C₂F₅CH═CHC₂F₅, (E-HFO-153-10mczz), C₃F₇CH═CHCF₃ (HFO-153-10mzz), (CF₃)₂CFCH═CHCF₃ (HFO-153-10mzzy), Z—C₂F₅CF═CFC₂F₅ (z-FO-151-12mcyZ) or a one of E-C₂F₅CF═CFC₂F₅(E-HFO-151-12mcyy), E-C₂F₅CH═CHC₂F₅, (E-HFO-153-10mczz), C₃F₇CH═CHCF₃ (HFO-153-10mzz), (CF₃)₂CFCH═CHCF₃ (HFO-153-10mzzy), Z—C₂F₅CF═CFC₂F₅ (z-FO-151-12mcyZ).

In use the thermal regulating medium is a fluoroolefin E-C₂F₅CF═CFC₂F₅ (E-HFO-151-12mcyy), E-C₂F₅CH═CHC₂F₅, (E-HFO-153-10mczz), C₃F₇CH═CHCF₃ (HFO-153-10mzz), (CF₃)₂CFCH═CHCF₃ (HFO-153-10mzzy), Z—C₂F₅CF═CFC₂F₅ (z-FO-151-12mcyZ) or blends thereof, directly (e.g., immersion) and/or indirectly contacting an electronic component, to regulate the thermal state of the electronic component, including but not limited to a battery, motor, computer, or server and/or an electrical component, or at least one of a battery, electric motor, electronics or heat pump system in one of a hybrid electric vehicle (HEV), mild hybrids electric vehicles (M HEV), plug-in hybrid electric vehicles (PHEV), or electric vehicles (EV). The method or system further includes at least one motive force for circulating the thermal regulating fluid to manage the thermal condition of the at least said one electrical component whether in the first and or second loops.

In certain embodiments thermal energy is exchanged between a working fluid and an electrical component by contacting an electrical component selected from one of a television, cell phone, monitors, drone, and avionics device; battery, powertrains for electronic vehicles, insulated-gate bipolar transistors (IGBTs), electronic devices-data center servers, computer server systems, telecommunication infrastructure, 5G network; displays, and military electronics where the working fluid comprises at least one fluoroolefins comprising E-C₂F₅CF═CFC₂F₅(E-HFO-151-12mcyy), E-C₂F₅CH═CHC₂F₅, (E-HFO-153-10mczz), C₃F₇CH═CHCF₃ (HFO-153-10mzz), (CF₃)₂CFCH═CHCF₃ (HFO-153-10mzzy), Z—C₂F₅CF═CFC₂F₅ (z-FO-151-12mcyZ), and the thermal conditions of the at least one component is regulated.

In certain embodiments thermal energy between a working fluid in a system selected from one of a high temperature mechanical vapor compression heat pump (HTHP), stationary air conditioning and chiller, Organic Rankine Cycle (ORC) containing and contacting a working fluid comprising at least one of fluoroolefins comprising E-C₂F₅CF═CFC₂F₅(E-HFO-151-12mcyy), E-C₂F₅CH═CHC₂F₅, (E-HFO-153-10mczz), C₃F₇CH═CHCF₃ (HFO-153-10mzz), (CF₃)₂CFCH═CHCF₃ (HFO-153-10mzzy), Z—C₂F₅CF═CFC₂F₅ (z-FO-151-12mcyZ) with the system and components of the system to thermally regulate the thermal condition of the system.

The system, further including at least one heat exchanging component for thermal exchange between the thermal regulating fluid and a device for circulating one of air, water, glycol based fluids, such as ethylene glycol or propylene glycol, or other fluid.

The system and method regulate the exchange of thermal energy between the thermal regulating medium and at least one electrical component requiring thermal management from one of a hybrid electric vehicle (HEV), mild hybrids electric vehicles (MHEV), plug-in hybrid electric vehicles (PHEV), or electric vehicles (EV) by circulating a thermal regulating fluid to directly/indirectly thermally contact at least one electrical component wherein said thermal regulating fluid comprises at least one of fluoroolefins comprising E-C₂F₅CF═CFC₂F₅ (E-HFO-151-12mcyy), E-C₂F₅CH═CHC₂F₅, (E-HFO-153-10mczz), C₃F₇CH═CHCF₃ (HFO-153-10mzz), (CF₃)₂CFCH═CHCF₃ (HFO-153-10mzzy), Z—C₂F₅CF═CFC₂F₅ (z-FO-151-12mcyZ) or blends thereof, which thermally regulates the thermal condition of said at least one component.

### Other Embodiments

Transferring thermal energy between a working fluid and an electrical component, comprising contacting an electrical component selected from one of:
a television, cell phone, monitors, drone, and avionics device; battery, powertrains for electronic vehicles, insulated-gate bipolar transistors (IGBTs), electronic devices-data center servers, computer server systems, telecommunication infrastructure, 5G network; displays, and military electronics, or
a high temperature mechanical vapor compression heat pump (HTHP), stationary air conditioning and chiller, and Organic Rankine Cycle (ORC),
with a working fluid comprising at least one of fluoroolefins comprising E-C₂F₅CF═CFC₂F₅(E-HFO-151-12mcyy), E-C₂F₅CH═CHC₂F₅, (E-HFO-153-10mczz), C₃F₇CH═CHCF₃ (HFO-153-10mzz), (CF₃)₂CFCH═CHCF₃ (HFO-153-10mzzy), Z—C₂F₅CF═CFC₂F₅ (z-FO-151-12mcyZ), and thermally regulating the thermal condition of said at least one component.

A system for transferring thermal energy between a working fluid and an electrical component, comprising contacting a component containing from one of:
a television, cell phone, monitors, drone, and avionics device; battery, powertrains for electronic vehicles, insulated-gate bipolar transistors (IGBTs), electronic devices-data center servers, computer server systems, telecommunication infrastructure, 5G network; displays, and military electronics, or
a high temperature mechanical vapor compression heat pump (HTHP), stationary air conditioning and chiller, and Organic Rankine Cycle (ORC), with
a working fluid circuit fluid comprising at least one of fluoroolefins comprising E-C₂F₅CF═CFC₂F₅ (E-HFO-151-12mcyy), E-C₂F₅CH═CHC₂F₅, (E-HFO-153-10mczz), C₃F₇CH═CHCF₃ (HFO-153-10mzz), (CF₃)₂CFCH═CHCF₃ (HFO-153-10mzzy), Z—C₂F₅CF═CFC₂F₅ (z-FO-151-12mcyZ) in energy exchanging contact with said component to thermally regulate the thermal condition of said one component.

A system for transferring thermal energy between a working fluid and an electrical component comprising or consisting essentially of a closed loop circuit containing as the working fluid, at least one of fluoroolefin comprising E-C₂F₅CF═CFC₂F₅ (E-HFO-151-12mcyy), E-C₂F₅CH═CHC₂F₅, (E-HFO-153-10mczz), C₃F₇CH═CHCF₃ (HFO-153-10mzz), (CF₃)₂CFCH═CHCF₃ (HFO-153-10mzzy), Z—C₂F₅CF═CFC₂F₅ (z-FO-151-12mcyZ), a motive force to circulate the working fluid, control valves to direct the working fluid to one or more electric components and collect working fluid from the one or more electrical components, and a member to exchange and recover energy from the working fluid.

A method for transferring thermal energy between a working fluid and an electrical component comprising or consisting essentially of a closed loop circuit containing as the working fluid, at least one of fluoroolefin comprising E-C₂F₅CF═CFC₂F₅ (E-HFO-151-12mcyy), E-C₂F₅CH═CHC₂F₅, (E-HFO-153-10mczz), C₃F₇CH═CHCF₃ (HFO-153-10mzz), (CF₃)₂CFCH═CHCF₃ (HFO-153-10mzzy), Z—C₂F₅CF═CFC₂F₅ (z-FO-151-12mcyZ), a motive force circulating the working fluid, directing the working fluid to contact one or more electric components and collecting working fluid from the one or more electrical components, and a recovering energy from the working fluid.

A method for transferring thermal energy between at least one of fluoroolefins comprising E-C₂F₅CF═CFC₂F₅ (E-HFO-151-12mcyy), E-C₂F₅CH═CHC₂F₅, (E-HFO-153-10mczz), C₃F₇CH═CHCF₃ (HFO-153-10mzz), (CF₃)₂CFCH═CHCF₃ (HFO-153-10mzzy), Z—C₂F₅CF═CFC₂F₅ (z-FO-151-12mcyZ) and an electrical component by immersion using the system of FIG. 2.

A method or system for transferring thermal energy by immersion by selecting working fluids which exhibit a dielectric constant suitable for electrical applications and exhibiting a low dielectric constant to provide increased electrical isolation of the electrical components, and immersing the electrical component in at least one of fluoroolefins comprising E-C₂F₅CF═CFC₂F₅ (E-HFO-151-12mcyy), E-C₂F₅CH═CHC₂F₅, (E-HFO-153-10mczz), C₃F₇CH═CHCF₃ (HFO-153-10mzz), (CF₃)₂CFCH═CHCF₃ (HFO-153-10mzzy), Z—C₂F₅CF═CFC₂F₅ (Z—FO-151-12mcyZ).

A method or system for transferring thermal energy by immersing the electric component in a tank comprising a dielectric immersion fluid including at least E-HFO-153-10mczz.

A method or system for transferring thermal energy by immersing the electric component in a tank comprising a dielectric immersion fluid including at least E-HFO-153-10mczz and having a dielectric constant over the operational frequency range (0 to 20 GHz) of less than 7.3, or less than 5.5, or less than 5.0, or less than 4.0, or less than 3.5, or less than 2.7, or less than 2.5, or less than 2.0, or less than 1.9, or less than 1.8, or less than 1.5.

The systems of at least ¶¶[0138]-[0140] further comprising a tank for holding the immersion fluid, and closed loop circuit for circulating a medium for controlling the temperature of the heated immersion fluid.

While the invention has been described with reference to one or more embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. In addition, all numerical values identified in the detailed description shall be interpreted as though the precise and approximate values are both expressly identified.

1. A method of producing a fluoroolefin comprising:
coupling 3,3-dichloro-1,1,1,2,2-pentafluoropropane (CF₃—CF₂—CHCl₂) in a liquid phase in the presence of a catalyst to form a composition comprising 1,1,1,2,2,5,5,6,6,6-decafluoro-3-hexene (CF₃CF₂CH═CHCF₂CF₃).

2. The method of claim 1, wherein the coupling occurs in an aprotic solvent.

3. The method of claim 2, wherein the aprotic solvent is selected from the group consisting of dimethylformamide, dimethylacetamide, dimethyl sulfoxide, and N-methylpyrrolidone.

4. The method of claim 1, wherein the catalyst is selected from the group consisting of 2,2-bipyridine, a copper(I) salt, and a combination thereof.

5. The method of claim 4, wherein the copper(I) salt is selected from the group consisting of CuCl, CuBr, CuI, and copper(I) acetate.

6. The method of claim 1, wherein the coupling occurs in the presence of copper powder.

7-22. (canceled)

23. A method of producing a fluoroolefin comprising:
coupling a compound of formula (1),
C₂F₅CHₙCl₃₋ₙ (1)
wherein n is 0 or 1;
in the presence of a first catalyst to form a composition comprising a compound of formula (2),
C₂F₅CX₁=CX₂C₂F₅ (2)
wherein when n is 0, X₁ and X₂ are Cl; and
wherein when n is 1, X₁ and X₂ are H, wherein n is 1 and X₁ and X₂ are H, and the coupling occurs in a liquid phase.

24. (canceled)

25. The method of claim 23, wherein the coupling occurs in an aprotic solvent.

26. The method of claim 25, wherein the aprotic solvent is selected from the group consisting of dimethylformamide, dimethylacetamide, dimethyl sulfoxide, and N-methylpyrrolidone.

27. The method of claim 23, wherein the first catalyst is selected from the group consisting of 2,2-bipyridine, a copper(I) salt, and a combination thereof.

28. The method of claim 27, wherein the copper(I) salt is selected from the group consisting of CuCl, CuBr, CuI, and copper(I) acetate.

29. The method of claim 23, wherein the coupling occurs in the presence of copper powder.

30-35. (canceled)

36. A composition formed by the method of claim 1.

37-42. (canceled)

43. A method for exchanging thermal energy with an electronic component comprising providing an electronic component and contacting a composition including one of HFO-153-10mczz fluoroolefins comprising E-C₂F₅CF═CFC₂F₅(E-HFO-151-12mcyy), E-C₂F₅CH═CHC₂F₅, (E-HFO-153-10mczz), C₃F₇CH═CHCF₃ (HFO-153-10mzz), (CF₃)₂CFCH═CHCF₃ (HFO-153-10mzzy), Z—C₂F₅CF═CFC₂F₅ (z-FO-151-12mcyZ), directly and/or indirectly with the electronic component to regulate the component temperature, wherein the fluoroolefin is obtained from the process of claim 1.

44-46. (canceled)

47. A method for transferring thermal energy, comprising providing at least one electrical component requiring thermal management from one of a hybrid electric vehicle (HEV), mild hybrids electric vehicles (MHEV), plug-in hybrid electric vehicles (PHEV), or electric vehicles (EV), circulating a thermal regulating fluid to directly/indirectly thermally contact at least one electrical component wherein said thermal regulating fluid comprises at least one of fluoroolefins comprising E-C₂F₅CF═CFC₂F₅ (E-HFO-151-12mcyy), E-C₂F₅CH═CHC₂F₅, (E-HFO-153-10mczz), C₃F₇CH═CHCF₃ (HFO-153-10mzz), (CF₃)₂CFCH═CHCF₃ (HFO-153-10mzzy), Z—C₂F₅CF═CFC₂F₅ (z-FO-151-12mcyZ), and thermally regulates the thermal condition of said at least one component, wherein the fluoroolefin is obtained from the process of claim 1.

48. (canceled)

49. A method for transferring thermal energy between a working fluid and an electrical component, comprising contacting an electrical component selected from one of:
a) a television, cell phone, monitors, drone, and avionics device; battery, powertrains for electronic vehicles, insulated-gate bipolar transistors (IGBTs), electronic devices-data center servers, computer server systems, telecommunication infrastructure, 5G network; displays, and military electronics, or
b) a high temperature mechanical vapor compression heat pump (HTHP), stationary air conditioning and chiller, and Organic Rankine Cycle (ORC)
with a working fluid comprising at least one of fluoroolefins comprising E-C₂F₅CF═CFC₂F₅(E-HFO-151-12mcyy), E-C₂F₅CH═CHC₂F₅, (E-HFO-153-10mczz), C₃F₇CH═CHCF₃ (HFO-153-10mzz), (CF₃)₂CFCH═CHCF₃ (HFO-153-10mzzy), Z—C₂F₅CF═CFC₂F₅ (z-FO-151-12mcyZ), and thermally regulating the thermal condition of said at least one component, wherein the fluoroolefin is obtained from the process of claim 1.

50. A system for transferring thermal energy between a working fluid and an electrical component, comprising a component containing from one of:
a) a television, cell phone, monitors, drone, and avionics device; battery, powertrains for electronic vehicles, insulated-gate bipolar transistors (IGBTs), electronic devices-data center servers, computer server systems, telecommunication infrastructure, 5G network; displays, and military electronics, or
b) a high temperature mechanical vapor compression heat pump (HTHP), stationary air conditioning and chiller, and Organic Rankine Cycle (ORC) and working fluid circuit fluid comprising at least one of fluoroolefins comprising E-C₂F₅CF═CFC₂F₅ (E-HFO-151-12mcyy), E-C₂F₅CH═CHC₂F₅, (E-HFO-153-10mczz), C₃F₇CH═CHCF₃ (HFO-153-10mzz), (CF₃)₂CFCH═CHCF₃ (HFO-153-10mzzy), Z—C₂F₅CF═CFC₂F₅ (z-FO-151-12mcyZ) in energy exchanging contact with said component to thermally regulate the thermal condition of said one component, wherein the fluoroolefin is obtained from the process of claim 1.

51. A method for transferring thermal energy between at least one of fluoroolefins comprising E-C₂F₅CF═CFC₂F₅ (E-HFO-151-12mcyy), E-C₂F₅CH═CHC₂F₅, (E-HFO-153-10mczz), C₃F₇CH═CHCF₃ (HFO-153-10mzz), (CF₃)₂CFCH═CHCF₃ (HFO-153-10mzzy), Z—C₂F₅CF═CFC₂F₅ (z-FO-151-12mcyZ) and an electrical component by immersion, wherein the fluoroolefin is obtained from the process of claim 1.

52. A method for transferring thermal energy by immersion by selecting fluids which exhibit a dielectric constant suitable for electrical applications and exhibiting a low dielectric constant to provide increased electrical isolation of the electrical components, and immersing the electrical component in at least one of fluoroolefins comprising E-C₂F₅CF═CFC₂F₅(E-HFO-151-12mcyy), E-C₂F₅CH═CHC₂F₅, (E-HFO-153-10mczz), C₃F₇CH═CHCF₃ (HFO-153-10mzz), (CF₃)₂CFCH═CHCF₃ (HFO-153-10mzzy), Z—C₂F₅CF═CFC₂F₅ (z-FO-151-12mcyZ), wherein the fluoroolefin is obtained from the process of claim 1.

53. The method of claim 52, wherein the dielectric working fluid including at least E-HFO-153-10mczz having a dielectric constant over the operational frequency range (0 to 20 GHz) of less than 7.3, or less than 5.5, or less than 5.0, or less than 4.0, or less than 3.5, or less than 2.7, or less than 2.5, or less than 2.0, or less than 1.9, or less than 1.8, or less than 1.5.

54. (canceled)
